(19) United States
(12) Patent Application Publication
(10) Pub. No.: US 2005/0025753 A1
(43) Pub. Date: Feb. 3, 2005

(54) Methods for production of non-disease causing hemoglobin by ex vivo oligonucleotide gene editing of human stem/progenitor cells

(76) Inventors: Wei Han, SongJiang (CN); Matthew J. Chomo, Wilmington, DE (US); Margaret Wong, Bear, DE (US); Barbara H. Fish, Wilmington, DE (US); Carolyn M. Ireland, Elkton, MD (US); Davette L. Behrens, Newark, DE (US)

Correspondence Address: Daniel M. Becker;c/o HELLER EHRMAN WHITE &, McAULIFFE LLP, 275 Middlefield Road, Menlo Park, CA, 94025, US

(21) Appl. No.: 10/837,013

(22) Filed: Apr. 30, 2004

Related U.S. Application Data
(60) Provisional application No. 60/475,941, filed on Jun. 4, 2003.
(60) Provisional application No. 60/467,234, filed on Apr. 30, 2003.

(51) Int. Cl. A61K 48/00; C12N 15/85

(52) U.S. Cl. 424/93.21; 435/455

(57) ABSTRACT

Methods are presented for applying ex vivo oligonucleotide gene editing to hematopoietic stem cells and/or progenitor cells to create therapeutically effective amounts of wild-type hemoglobin for treatment of the hemoglobinopathies.

### CROSS-REFERENCE TO RELATED APPLICATIONS

This application claims the benefit of U.S. provisional application Nos. 60/475,941, filed Jun. 4, 2003, and 60/467,234, filed Apr. 30, 2003, the disclosures of which are incorporated herein by reference in their entireties.

### BACKGROUND OF THE INVENTION

The hemoglobinopathies are a major source of morbidity and mortality in the United States and worldwide. Yet despite their prevalence, current treatments are few and imperfect.

For sickle cell anemia (“SCA” or other forms of Sickle Cell Disease (“SCD”), for example, there is at present only a single drug, hydroxyurea, for treating the underlying hematologic disorder which is effective in reducing the frequency of episodes of vaso-occlusive crises, acute chest syndrome, and hospitalizations. However, hydroxyurea can cause neutropenia and thrombocytopenia, placing patients respectively at risk for infection and bleeding.

Repetitive transfusion to treat the anemias resulting from the beta and alpha hemoglobinopathies can lead to iron overload and communication of infectious disease, typically viral.

Bone marrow transplant, while curative when successful, depends for its success upon the availability of an adequately matched donor and an appropriate ablative marrow procedure. Complications include death from complications of the ablation procedure, infection, nonengraftment, and graft-versus-host disease.

Diseases of the hematopoietic system have long been considered in theory the best candidates for ex vivo gene therapy, particularly with the use of viral vectors (e.g., retroviruses, adenoviruses, AAV viruses and lentiviruses). Ex vivo viral vector gene therapy produces a self-renewing population of hematopoietic stem and/or progenitor cells containing a normal hematopoietic globin transgene. The hematopoietic stem and/or progenitor cells can be obtained by leukophoresis (e.g., enrichment of white blood cells from peripheral blood), bone marrow biopsy or mobilization to the periphery; the selectively enriched population of cells containing stem/progenitor cells can readily be cultured and transfected; and the transfected cells then readily returned for engraftment. The very first documented human gene therapy experiment, in 1980, was intended to treat thalassemia; the first approved human gene therapy experiment in 1989 was designed to treat ADA-SCID.

Nonetheless, experience with ex vivo gene therapy of blood disorders over the past two decades has been disappointing, and recent reports of leukemia in two ADA-SCID patients otherwise cured by gene therapy has cast serious doubt on the viability of retrovirus-mediated approaches. Similarly, the death of one male teenage recipient of an adenoviral gene therapy procedure in Pennsylvania also raises serious concerns about viral vectors and immunological responses to viral vector gene therapy procedures. Similarly, the recombination and insertion into chromosomes by viral vectors or large polynucleotides have raised concerns about the effect of gene interruption and the mutagenic consequences of recombination based approaches of gene conversion.

Recently, methods for oligonucleotide-mediated targeted gene editing involving repair of a targeted microlesion, usually a single nucleotide, in a genomic chromosomal or episomal DNA have been developed. In these approaches, chimeric RNA/DNA oligonucleotides (e.g., Kmiec type chimeric vectors, see, e.g., U.S. Pat. Nos. 5,565,350; 5,731,181, 5,795,972 and 5,888,983, the disclosures of which are incorporated herein by reference in their entireties) or modified single-stranded oligonucleotides introduced into the cell mobilize the cellular machinery and certain cellular repair proteins in the cell to alter the targeted genomic sequence to that borne by the oligonucleotide. See, e.g., Agarwal et al., “Nucleotide replacement at two sites can be directed by modified single-stranded oligonucleotides in vitro and in vivo,” Biomol Eng. 20(1):7-20 (2003); Pierce et al., “Oligonucleotide-directed single-base DNA alterations in mouse embryonic stem cells,” Gene Ther. 10(1):24-33 (2003); Liu et al., “Targeted beta-globin gene conversion in human hematopoietic CD34+ and CD38− cells ”, Gene Ther. 9: 118-126 (2002); Cole Strauss et al., “Correction of the mutation responsible for sickle cell anemia by an RNA/DNA oligonucleotide”, Science 273: 1386-1389 (1996); WO 02/10364; and WO 01/73002, the disclosures of which are incorporated herein by reference in their entireties.

The gene editing approach makes no genetic changes elsewhere than at the targeted locus, and introduces no viral genomic or protein elements (the latter having the capability of eliciting an adverse immunologic response in a mammal or patient), offering a potential solution to the problems inherent in viral-mediated gene therapy approaches.

Regardless of the success of the genetic conversion event resulting from the gene editing of a target cell population, production of normal or non-disease causing hemoglobin has not heretofore been demonstrated in a mammal, including a human or a human cell, using a gene editing procedure. There thus exists a need in the art for methods of applying ex vivo oligonucleotide based gene editing to hematopoietic stem cells and/or progenitor cells to produce non-disease causing human hemoglobin (e.g., hemoglobin which results in no disease produced from normal wild type globin genes, alleles of the normal globin genes, or other alleles of the globin genes which have been recognized to ameliorate the effects of the defective hemoglobin which causes disease) or to create therapeutically effective amounts of wild-type hemoglobin for treatment of hemoglobinopathies.

### SUMMARY OF THE INVENTION

The present invention solves these and other needs by providing a mammal having a hemoglobinopathy, including a human, with stem/progenitor cells which have been subjected to a targeted nucleotide exchange in the defective chromosomal gene causing the hemoglobinopathy. The stem/progenitor cells are allowed to engraft the mammal, including a human. Thereafter, the mammal, including the human, produces at least two detectable populations of hemoglobin. Alternatively, the mammal, including the human, produces only hemoglobin from the gene edited stem/progenitor cells provided to the mammal, including a human, in the ex vivo procedure as a result of additional interventional procedures such as myeloablative marrow procedures using irradiation or drugs and re-engraftment of gene edited cells. Provision of multiple oligonucleotides designed to simultaneously target more than one mutant disease causing nucleotide may produce more than two detectable populations of hemoglobin because multiple mutant disease causing alleles may not be corrected simultaneously in the same cell. In this embodiment, multiple oligonucleotides directed to altering more than a single nucleotide in a hemoglobin gene are used in the gene editing procedure and alter one or more nucleotides in the stem/progenitor cells. In addition to the production of hemoglobin from the gene possessed at birth, additional hemoglobin species are produced for each type of oligonucleotide directed alteration. Absent some type of myeloablative intervention prior to engraftment of the gene edited stem/progenitor cells, the predominant population is the disease causing hemoglobin the individual mammal, including a human, produced at birth. The additional populations of hemoglobin are the non-disease causing hemoglobin(s) produced in red blood cells arising from differentiation of the altered hematopoietic stem/progenitor cells provided to the mammal. Sufficient amounts of the non-disease causing hemoglobin are produced to alleviate at least one of the disease symptoms resulting from the hemoglobinopathy.

### DETAILED DESCRIPTION

Hemoglobin and Hemoglobinopathies

Hemoglobin (Hb) is the respiratory pigment essential for human life as the oxygen transporter to tissues and constitutes about 90% of the dry weight of the average red blood cell. Hemoglobin also functions to transport carbon dioxide and provide a buffering action to maintain pH within a normal range. Hb interacts with three diffusible ligands: O2 (oxygen), CO2 (carbon dioxide) and NO (nitric oxide). A normal hemoglobin molecule (Hb A) is a tetramer composed of two dissimilar pairs of polypeptide chains (alpha2beta2), each of which encloses an iron-containing porphyrin designated heme. In adults, 96-98% of hemoglobin is Hemoglobin A which has two alpha chains and two beta chains. Properties of normal adult hemoglobin are that it is soluble in both oxy and deoxy states; it is stable; it binds oxygen reversibly with P50 of about 27 torr; it maintains iron in the reduced ferrous state (Fe2+) in the heme moiety; and it contains balanced amounts of alpha and non-alpha globin polypeptide chains. Hb stability refers to its ability to maintain its quaternary structure due to stable and coordinated intramolecular bonds amongst its subunits (alphalbeta2 and alphalbetal contacts). Mutations at the alphalbeta2 interface are usually associated with changes in the oxygen affinity of hemoglobin, whereas mutations at the alphalbetal interface usually cause hemoglobin instability. Normally the four heme groups do not undergo oxygenation or deoxygenation simultaneously, but sequentially, depending on the state of each individual heme unit, with regard to the presence or absence of bound oxygen on the other three globin chains. Normally, the P50 or the oxygen tension at which Hb is half saturated is about 27 torr (mmHg). Increased P50 indicates decreased oxygen affinity and vice-versa. Normally the ratio of alpha globin to non-alpha globin is about 1. Significant changes in this ratio result in surplus intracellular globin chains that interfere with normal function and cellular survival. This aberration in the alpha/non-alpha ratio is the hallmark of thalassemia.

Derangement in any of the foregoing properties of normal hemoglobin impedes effective oxygen transport and usually leads to clinical disorders which cause disease. Abnormal properties are insolubility which produces sickle cell syndromes; instability which produces congenital hemolytic anemia; abnormal oxygen affinity which produces familial erythrocytosis (polycythemia); oxidized heme which produces M hemoglobins; and unbalanced alpha/beta synthesis which produces thalassemia (Hb E).

A single mutation in the 6^{th} th codon of exon 1 of the beta-globin gene (GAT to GTT) responsible for the synthesis of the beta-globin chain is the cause of sickle cell disease. The normal glutamic acid is replaced by valine at position 6 in the beta chain producing sickle hemoglobin (Hb S) and results in a +1 charge as compared to Hb A. Sickle cell anemia is the homozygous state (SS). Other sickle cell syndromes result from the co-inheritance of the sickle gene and a non-sickle gene such as Hb C, Hb Oarab, Hb D, B+-thalassemia, beta0-thalassemia, etc. Major types of sickle cell syndromes and their typical hematological parameters are presented in Table 1 (provided as a separate sheet).

Major hematological manifestations of sickle cell anemia are normochromic, normocytic anemia with a mean Hb of 7.8 plus or minus 1.13 and a mean corpuscular volume (MCV) of 90 fl. The presence or absence of alpha-gene deletion has an effect on the anemia, the indices and the hemoglobin electrophoresis pattern. Thus, patients with sickle cell anemia and homozygous alpha-thal 2 (betaSbetaS; -alpha/-alpha) have milder anemia, a lower reticulocyte count, a low MCV and a high Hb A2 level. Both the white blood cell and platelet counts are increased in SCA due to increased marrow activity secondary to chronic hemolysis, and platelets are not stored in the spleen.

The acute painful sickle cell crisis is the hallmark of sickle cell anemia and is the most common complaint among patients with this disease. Severe painful episodes necessitate treatment in the emergency room and/or hospital (over 90% of hospital admissions of adult patients). Objective signs of a painful crisis are fever, leukocytosis, joint effusions and tenderness. Serial determinations in the evolution of the painful crisis show that the percentage of irreversibly sickled cells or dense cells is high early in the crisis and decreases gradually as the crisis evolves. About 10% of patients have over 20 crises per year. Bone marrow infarcts are associated with severe pain. Leg ulceration occurs in 5-10% of adult patients and are more common in males, less common in patients with alpha-gene deletion, occur more frequently in older patients, less in patients with high total Hb levels, and less in patients with high levels of Hb F.

Although individuals with sickle cell trait are resistant to infection with Plasmodium fulciparum, SCA patients are susceptible to infection by Plasmodium falciparum. Individuals with Fy(a-b-) Red Blood Cells (RBC) are resistant to infection by other types of malarial parasites. SCA patients have increased susceptibility to infection with polysaccharide-encapsulated bacteria (S. pneumoniae and H. influenzae). Transfusion related iron overload and abnormalities in B cell immunity may explain antigen processing defects. Infections with E. coli are usually associated with urinary tract infection in adults. SCA patients are susceptible to osteomyelitis infections of S. typhimurium and S. aureus.

CNS complications occur in 25% of patients with sickle cell disease. Intracerebral hemorrhage is prevalent in adults and microaneurysms involving fragile dilated vessels around areas of infarction seem to be responsible for hemorrhage in adults.

Acute chest syndrome is relatively frequent in SCA and includes chest pain, fever, dyspnea, hypoxia, pulmonary infiltrates on chest x-ray, and a decreasing Hb level.

Beta-thalassemia is a disorder characterized by absent or diminished beta chain synthesis. In sickle-beta-thalassemia, both beta-globin genes are defective, one producing an abnormal beta chain and the other affecting the rate of beta chain synthesis.

Sickle-C-Disease (Hb SC disease) with Hb C (alpha2beta2 with 6glu to lys and a charge compared to normal of +2) occurs with a frequency about a quarter of that for Hb S. However SC disease is almost as common among adults as SS disease since life expectancy in SC disease is nearly normal. Intracellular hemoglobin concentration is higher is SC red cells due to Hb C and SC red cells have at least a 10% higher level of Hb S than sickle trait (AS) patients have.

Homozygous Hb C disease produces a mild congenital hemolytic anemia with splenomegaly. Hb C is less soluble than Hb A and it tends to form intracellular crystals.

M hemoglobins include alpha2 58his to tyr beta2; alpha2 87 his to tyr beta 2; alpha2 beta2 63his to tyr; alpha2beta2 92 his to tyr and alpha2beta2 67 val to glu. Disease symptoms include cyanosis.

Hemoglobin E is a beta chain variant (alpha2beta2 26glu to lys) common in South East Asia. The beta chain is synthesized at a reduced rate compared with beta A due to a false splicing site within an exon producing both normal and abnormal splicing. Abnormal spliced mRNA transcripts are processed abnormally. Co-inheritance of Hb E with beta-thalassemia trait leads to thalassemia major or thalassemia intermedia. Homozygosity for Hb E results in a clinically mild condition.

Beta thalassemia minor or trait results in minimal globin chain imbalance and anemia on the basis of inheritance of a single beta thal gene. Beta thalassemia intermedia results in moderate globin chain imbalance and moderate anemia on the basis of inheritance of two beta thal genes. Beta-thalassemia major or Cooley's Anemia results from profound globin chain imbalance and severe anemia on the basis of inheritance of two beta-thal genes. A beta0-thalassemia gene is a mutant gene which does not result in the synthesis of any normal beta globin. Beta+ thalassemia gene is a mutant which results in decreased synthesis of beta globin.

Production of Non-Disease Causing Hemoglobin:

Selectively Enriched Cells

Hematopoietic stem/progenitor cells (HS/PC) are present in selectively enriched peripheral blood or bone marrow preparations isolated from a mammal, including a human, or alternatively include cultured human embryonic stem cells, which may be derived e.g., from human embryos or oocytes. In a preferred embodiment for correction of sickle cell anemia, the cells are obtained from peripheral blood by leukophoresis of a mammal, including a human, during the period of an acute crisis. The HS/PC cells are present in CD34+ enriched cells using, for example, either the Miltenyi or Isolex methods of enrichment or its equivalent. See, e.g., the Miltenyi CD34+ progenitor cell isolation kit using indirect magnetic labeling for human CD34+ precursor cells from peripheral blood, bone marrow and/or cord blood at www.miltenyibiotec.com and protocols contained therein; see the Baxter Isolex system in e.g., Rowley et al, Bone Marrow Transplantation, 21(12): 1253-1262 (June 1998) which describes the immunomagnetic separation technique to enrich human CD34+ cells from peripheral blood stem cell components or bone marrow in a human trial following mobilization chemotherapy with g-CSF; a comparison of two systems is described by J. McMannis et al., permanent abstract 016 at www.celltherapy.org/ABS2000/posterpresentations characterized by the key words “CD34 Cell Selection, Isolex, CliniMACS, T-cell depletion” in which the authors concluded there was no statistical difference in purity or yield although B cell depletion was greater for Isolex due to a negative selection step. A commercial source of selectively enriched normal CD34+ human cells may be obtained from e.g., Whittaker.

Following selective enrichment or after oligonucleotide introduction, the cells may be cultured for up to four days pre-oligonucleotide introduction or for up to 9 weeks post-oligonucleotide introduction in appropriate medium, e.g., Iscove's Modified Dulbecco's medium (IMDM) medium (available from Gibco/Invitrogen, Mediatech, or Sigma) containing either 10% BIT 9500 Serum Substitute from Stem Cell Technologies or containing 10% Fetal calf serum, penicillin/streptomycin (pen/strep) at 25 U/25 micrograms/ml, and monothioglycerol (obtained from e.g., Fisher Scientific). For cells, PeproTech Inc. cytokines are included at 100 nanograms each per ml medium; appropriate human or mouse cytokines added to homologous cells are recombinant human or mouse stem cell factor, recombinant human or mouse flt3-ligand and recombinant human or mouse thrombopoietin. Alternatively, non-recombinant human cytokines of GMP quality may be used with human cells. For proliferation and differentiation of erythroid progenitor cells, appropriate human or mouse erythropoietin is included at 1 U/ml. Tissue culture grade recombinant human Epo can be obtained from, e.g., R&D Systems, Inc. Addition of erythropoietin results in cells having differentiated characteristics and properties of BFU-E, CFU-E and CFU-G/M.

In one embodiment using an animal model, bone marrow and/or blood from transgenic mice may be used as the source for selective enrichment of CD34+ cells. The transgenic mice express human sickle hemoglobin, such as those described in Paszty et al., Science 278: 876-878 (October 1997). Dr. T. Asakura of the Children's Hospital of Pennsylvania is the source of the transgenic mice used in these experiments, which are the progeny of mice created by Dr. Mohandas, a co-author of Paszty et al. Because the hemoglobins produced by these mice can be differentiated into mouse and human forms, including sickle and normal, using the HPLC procedure, heterozygous mice containing both mouse and human genes can be used in the gene editing experiments. In this embodiment, varying numbers of selectively enriched cells from one or more transgenic mice are gene edited as described herein and re-introduced into other irradiated siblings to transplant the edited CD34+ cells. At various times thereafter, samples from the transplanted mice are analyzed to analyze the different populations of hemoglobin produced following transplantation. Correlations between the number of edited transplanted cells, the number of transplanted cells in which the target nucleotide is converted, the production and amounts of normal human hemoglobin, and the amounts of the various hemoglobins define the optimal number of gene edited converted HS/PC cells necessary for engraftment to produce normal human hemoglobin in the transgenic mice model. Moreover, those numbers can also be correlated with the alleviation of symptoms of sickle cell disease in appropriate heterozygous and homozygous mice, which symptoms include anemia, average hematocrits in the 65% range for homozygous sickle mice, elevated reticulocyte counts, in vitro sickling upon deoxygenation, decreased osmotic fragility, increased dynamic rigidity and damage to multiple organs exemplified by increases in spleen, heart and kidney weights and histologic pathologies.

Ex Vivo Oligonucleotide Introduction to Enriched Cells:

Genotypic Conversion of a Globin Gene

The oligonucleotide, as described in different embodiments herein, is introduced by electroporation or transfection into mammalian CD34⁺ cells that are isolated from peripheral blood or bone marrow or equivalent source in the mammal, including the human. Oligonucleotide uptake and kinetics in the enriched CD34⁺ cells or cultures of human or mouse embryonic stem cells are determined by measuring uptake of FITC conjugated oligos using fluorescent activated cell sorting (FACS). In a preferred embodiment, the cells are cultured as described herein for up to four days before electroporation.

Cultured mammalian enriched cells, e.g., mouse, human, primate cells, are washed and concentrated and transferred into electroporation medium if oligonucleotide is to be introduced by electroporation. Electroporation medium for human cells is IMDM containing human serum albumin at 1% final concentration, pen/strep at 25 U/25 microgram/ml final concentration and monothioglycerol. For mouse cells, Iscove's Medium is used. Alternatively, electroporation is accomplished with cells in Cytofusion Medium Formula C from CytoPulse Sciences, Inc. Oligonucleotide may also be introduced into the cells using e.g., the Amaxa nucleofector kit with solution supplement 1. Electroporation is accomplished using the Square Wave Electroporation device. Alternatively, electroporation using MaxCyte or CytoPulse Sciences systems may be employed. For electroporation, cells are at a concentration of approximately 0.25 to 10 million cells per ml in a volume of 500 microliters using a 4 mm gap in the Square Wave system. In a preferred embodiment the cells are between 0.5 and 4 million cells per ml. In the most preferred embodiment, the cells are approximately 1 million cells per ml. Under these conditions, viability is optimally between 40 and 95% of input non-electroporated control cells using the Square Wave device. In a preferred embodiment, viability is between 80-95% of input non-electroporated cells.

For electroporation, voltages of 220-300V with a pulse of 38 ms produces viable cells following electroporation and having exchange of the targeted nucleotide and capable of producing hemoglobin in differentiated cultures. In a preferred embodiment, electroporation uses 240-280 V. Between 20 and 250 micrograms of oligonucleotide is used per 500 microliters. In one embodiment, oligonucleotide is provided between 30 to 90 micrograms per 500 microliters. .In a preferred embodiment, oligonucleotide is between 30 and 60 micrograms per 500 microliters of cells.

Oligonucleotide may also be introduced into cells using standard lipofection procedures, described herein in references incorporated by reference.

A single stranded oligodeoxynucleotide of 17 to 121 nucleotides having an internally unduplexed (non-hairpin) contiguous domain of at least 8 contiguous deoxyribonucleotides and either with or without terminal modifications (including 5′ or 3′ modifications at or near the terminus which include e.g., a phosphorothioate modification, an 2′o-methyl or ethyl modification, an LNA modification or lacking a modification; see, e.g., WO 01/73002 herein incorporated by reference in its entirety; see also U.S. Pat. No. 6,271,360 herein incorporated by reference in its entirety), or a Kmiec type chimeric internally duplexed doublestranded RNA/DNA oligonucleotide having double hairpins of about 71-74 nucleotides in length and with a 5′ and 3′ end such that it is not a covalently closed circular molecule, is synthesized and purified. Alternatively, oligonucleotides may be single strands of short restriction fragments up to 250 nucleotides in length or synthesized oligonucleotides of up to 250 nucleotides in length. In a preferred embodiment, the oligonucleotide targets one base in the genomic sequence of one of the human globin genes, e.g., spanning the point mutation (“T”) present in protein codon 6 of the sickle allele which encodes (β^{s}) and converts the normal glutamic acid to valine or it may target the mutant nucleotide of one of the globin genes responsible for thalassemia (see, e.g., oligonucleotides corresponding to those having the nucleic acid sequences corresponding to Seq ID numbers 357 through 500 and Seq ID numbers 2776 through 2979 or portions thereof that correspond to the nucleotide targeted for change in Tables 12, 22 and 23 of WO 01/73002, incorporated by reference herein in its entirety. In one embodiment, the oligonucleotide has an internally unduplexed domain of at least 8 contiguous deoxyribonucleotides and is fully complementary in sequence to the sequence of a first strand of the nucleic acid chromosomal/genomic target but for one or more mismatches as between the sequences of said deoxyribonucleotide domain and its complement on the target nucleic acid first strand, each of said mismatches positioned at least 8 nucleotides from said oligonucleotide's 5′ and 3′ termini and having a DNA portion required for nucleotide exchange that is identified by the Seq ID numbers 357 through 500 in Table 12 described above or in Seq ID numbers 2776 through 2979 in Tables 22 and 23 described above. In preferred embodiments, the oligonucleotide has modifications on at least the 3′ terminus. In additionally preferred embodiments, the modification on the 3′ terminus is at least one phosphorothioate modification, a 2′-o-methyl base analog or comprises an LNA backbone modification. In the most preferred embodiments, there are at least three modifications on at least the 3′ terminus and there may be additional modifications on the 5′ terminus. Alternatively, the oligonucleotide may be a chimeric RNA/DNA oligonucleotide of the type invented by E. Kmiec and colleagues and having an internally duplexed DNA portion required for nucleotide exchange that is comparable to portions of the Seq ID numbers 357 through 500 in Table 12 described above or Seq ID numbers 2776 through 2979 in Tables 22 and 23 described above which are centered around the described and underlined mismatch nucleotide. The oligonucleotide further may be labeled with a detection moiety such as fluorescein, e.g. FITC, for uptake and stability analyses. In one embodiment, single stranded oligonucleotides as described above having three phosphorothioates at each termini are between 21 and 71 nucleotides long. In a preferred embodiment the oligonucleotide is between 51 and 71 nucleotides long. In a separate embodiment, more than one type of oligonucleotide may be used, e.g., an oligonucleotide having a DNA portion that is up to 121 nucleotides long surrounding the sickle base T in the 6^{th} codon of the beta globin protein encoded by bases corresponding Seq ID numbers 357 through 360 of Table 12 described above may be used in conjunction with an additional oligonucleotide designed to correct another base in one of the globin genes encoding for example a thalassemia mutation, which may be encoded, for example, by Seq ID numbers 361 through 500 and Seq ID numbers 2776 through 2979 described above.

Transfected or electroporated CD34⁺ cells may be cultured for days to weeks after electroporation to define longterm viable cells that are colony forming and capable of production of red blood cells upon stimulation with erythropoietin.

To determine nucleotide conversion, for example of the sickle mutation (either normal to sickle or sickle to normal depending on the initial genetic constitution of the cells used) after transfection or electroporation, DNA is extracted and PCR used to amplify a targeted portion of the globin gene, e.g., a 352 bp region of beta-globin gene flanking codon 6 if the oligonucleotides are designed to convert the sickle gene to a normal allele or vice versa in the normal to sickle conversion. Conversion of the allele is seen at a nucleotide exchange frequency of between 0.1% up to 15% or higher and including 0.2%, 0.3%, 0.5%, 1%, 1.5%, 2%, 3%, 4%, 5%, 6%, 7%, 8%, 9%, 10%, 12%, 14%, 20% and 25% or higher as determined by topo cloning immediately following electroporation. Alternatively, colony forming cells are followed in a methylcellulose cell culture assay and screened at various times following oligonucleotide introduction into cells. In various experiments converted CFC cells are seen at a frequency of between 1 to 10% in different experimental conditions. Alternatively, cells are introduced into NOD-SCID mice, allowed to engraft, and the NOD-SCID mice are examined at various times after transplantation to determine engraftment and the numbers of SCID repopulating cells which is a measure of the stem/progenitor characteristics of the gene edited cells. Similarly, the DNA of the various cell populations is analyzed by sequencing, by SnaPshot (ABI Prism kit from Applied Biosystems, Foster City Calif.) or by fluorogenic 5/nuclease assay using TaqMan allele-specific amplification in the presence of two distinct fluorescent-labeled (VIC and FAM) probes complementary to the normal and sickle sequences.

Red blood cell differentiation in culture results in the production of at least two populations of hemoglobin, depending on the number of mutations targeted and the number of oligonucleotides used in the gene editing procedure. See FIG. 1 which demonstrates detection of two populations of hemoglobin in a two week differentiated culture of normal human cells able to produce red blood cells and hemoglobin in those cells in which a 71 nucleotide long oligonucleotide directs the conversion of the normal globin gene to a sickle gene. As a result of the gene editing process, two populations of hemoglobin are produced: Hb S and Hb A. In these experiments using FACS analysis, cells were stained with FITC labeled monoclonal antibodies specific for Hb A (e.g., lot 111419 of Perkin Elmer product code MBA-F) or for Hb S (e.g., lot 142986 for product code MBS-F). Cells were prepared using a BD Biosciences cell fixation/permeabilization kit (Cytofix/Cytoperm Kit). Hb S is labeled in the upper portion of the figure as Hb S FITC and present in 0.2% to about 13% of total hemoglobin depending on whether the cells were precultured for none, one, two or three days. The control is mouse IgG1 stained with FITC in this figure. In the lower portion of the figure, Hb A is present in 73 to about 85% of the cells. Thus Hb S is produced by cells in which the globin gene has been edited to produce the sickle mutation and Hb A is produced by other cells in the same culture which were not edited.

In an alternative embodiment, hemoglobin is analyzed using high performance cation-exchange chromatography. See, e.g., Mario et al, Clinical Chemistry 43(11): 2137-2142 (1997) for methods to differentiate Hb A, Hb F, Hb A2, Hb S, Hb C and Hb E. Slight differences are described for Hb C-Harlem and Hb D-Punjab variants. In general, in this procedure, an integrated HPLC System Gold from Beckman having a model 126 pump gradient, a Model 166 UV/visible detector set at 418 nm, a Model 507E autosampler, and System Gold software (vers. 8.1) or equivalent is used. The system is equipped with a 100 ×4.0 mm column packed with a weak cation-exchanger, porous (100 nm pore size) 5 micrometer microparticulate polyaspartic acid-silica (Poly Cat A) purchased from Touzart & Matignon (France) or equivalent. Separation of the hemoglobins is accomplished for example, by a salt gradient obtained by mixing buffers A (Bis-Tris 20 mmol/L, KCN 2 mmol/L, pH 5.8) and buffer B (Bis-Tris 20 mmol/L, KCN 2 mmol/L, sodium citrate 75 mmol/L, pH 5.8). The flow rate is approximately 1.5 ml/min. The column is equilibrated with B:A 33:67 by volume. After injection of sample, the proportionof B is increased linearly to B:A (45:55 by volume) and to 100:0; the mobile phase is returned to 33:67 for reequilibration.

Identification of human stem cells is monitored by transplantation of cells that are gene edited and demonstrate a single nucleotide conversion or not by repopulation using direct injection of primitive human hematopoietic cells into NOD/SCID mice bone marrow. See for example, the procedures described by Yahata et al, Blood 101(8): 2905-2913 (2003). For example, NOD/SCID mice are sublethally irradiated with 275 Cgy (dose rate 50 cGy/min) in a cesium irradiator or its equivalent. Twenty-four hours after irradiation, gene edited CD34+ cells as described herein are injected, for example via the tail vein. Mice are sacrificed at various times, bone marrow is collected. Samples are stained with various antibodies to detect multilineage engraftment of human cells using appropriate isotype-control antibodies.

Similarly, in therapeutic use of the cells which are subjected to gene editing ex vivo, upon introduction of cells back into the individual mammal or patient, and/or engraftment of those cells, normal hemoglobin as described above is produced at a frequency of at least 0.1% or higher, including 0.2%, 0.3%, 0.5%, 0.75%, 1%, 1.5%, 2%, 3%, 4%, 5%, 6%, 7%, 8%, 9%, 10%, 12%, 14%, 16%, 18%, 20%, 23%, 26%, 30%, 35%, 40% or higher of total hemoglobin. As a result the hemoglobin in a culture or in the individual mammal or patient comprises at least two populations: the disease causing hemoglobin defined by the individual at birth and the hemoglobin(s) produced by the gene edited cells.

Nucleotide exchange is monitored at the genetic level by PCR analysis of products which are cloned into plasmids using a TA topo cloning kit, transformed into bacteria and sequenced to define the altered nucleotide. Generally, 96 bacterial colonies are picked into a 96-well plate and subjected directly to allele-specific SNaPshot sequencing analysis to detect the targeted single base pair change, e.g., A to T at the codon 6 position for oligonucleotides designed to correct the sickle mutation in a human or the appropriate nucleotide for correction of other hemoglobinopathies as described elsewhere herein.

(1) We achieve between 50 to 95% oligonucleotide uptake in CD34⁺ cells. Voltage and duration of the electroporation pulse and the pre-culture/stimulation of CD34⁺ cells affect oligonucleotide uptake. Interestingly, oligonucleotide uptake dropped very quickly to below 10% 18 hrs post-electroporation.

(2) We achieve targeted single base pair change in 0.2% up to 7-15%, including 0.5%, 1%, 2%, 3%, 4%, 5%, 6%, 7%, 8%, 9%, 10%, 12%, and 15% of cells as determined by topo cloning which does not differentiate between stem and progenitor cells. In other embodiments, we achieve targeted single base pair change in 1-5% of colony forming cells which can survive for multiple weeks in cell culture indicating they are stem cells. In another embodiment, SCID repopulating cells are identified and sequenced to define the conversion frequency in that cell population. In one embodiment of electroporation conditions, 3.6% of CD34⁺ cells are demonstrate the targeted alteration. In an additional embodiment, 5.7% of CD34+ cells demonstrate the targeted alteration. In other embodiments, higher percentages of CD34+ cells demonstrate the targeted alteration.

In one embodiment, two populations of human hemoglobin are produced as described above. We also discover that multiple factors affect the efficiency of the gene correction including: oligonucleotide length, modification, and quality; and pre-culture/stimulation of CD34⁺ cells. In a preferred embodiment the oligonucleotide is 51 to 71 nucleotides long; in a more preferred embodiment the oligonucleotide is 61 nucleotides long and has three phosphorothioates on each termini. In one embodiment with a 71 long oligonucleotide, a single terminal LNA modification on each of the 5′ and 3′ ends was preferred as compared to the same oligonucleotide sequence and length having three phosphorothioate modified termini.

We have thus developed a clinically applicable protocol using synthetic oligonucleotides to achieve allele specific gene editing of the beta globin gene in hematopoietic stem or progenitor cells which results in the production of at least two populations of hemoglobin. The production of the non-disease causing hemoglobin is sufficient to alleviate the hematological manifestations of the disease caused by the globin mutation of the animal, including a human. The therapeutic effectiveness can be screened by any one of the following methods: an increase in the g/dL of normal Hb, preferably Hb A; an increase in the hematocrit of the individual following treatment, which if a human the increase results in a hematocrit in the range of 28-38%; a decrease in the reticulocyte count; an increased interval between transfusions of normal blood used to treat the patient; a decrease in hospital admission of a patient population; a decrease in infection by a viral or bacterial pathogen; a decrease in acute painful episodes; a decrease in chronic pain syndromes; a decrease in dactylitis; a decrease in priapism in males; a decrease in cerebral infarct; a decrease in CNS hemorrhage; a decrease in the incidence of acute chest syndrome in a population of patients; a decrease in leg ulcer size, number and frequency; a change in the mean corpuscular volume of a red blood cell.

### Figure Legends

FIG. 1. Hemoglobin produced by cultured cells is analyzed as described herein using labeled antibodies. The samples are derived from cells that are not pre-cultured (d0 Ep), or cultured for 1, 2 or 3 days prior to electroporation (respectively d1 Ep, d2 Ep, d3 Ep). The three FITC labeled antibodies are for mouse IgG (control), normal human hemoglobin (Hb A) or human sickle hemoglobin (Hb S) following a normal to sickle conversion of human CD34+ gene edited cells.

FIG. 2. Detection of single base changes by SnaPshot and sequencing. The ABI prism SnaPshot Multiplex Kit from Applied Biosystems was used to detect the single base change at the SCA mutation site. A PCR amplified fragment flanking codon 6 of the beta-globin hemoglobin chain served as a template for an unlabeled oligonucleotide primer. In the presence of fluorescent labeled ddNTPs and AmpliTaq DNA polymerase, the primer was extended by one base pair, adding a ddNTP to its end at the target bse. The base change was detected after electrophoresis and analysis by GeneScan software. The figure illustras a normal (A) to sickle (T) conversion in one of the beta globin alleles. The second allele remains normal, hence the presence of both T and A in the sample. To detect gene conversion by sequencing, BigDye Terminator v3.1 Cycle Sequencing Kit (Applied Bisystems, CA) is used. The figure shows a normal to sickle conversion (A to T) in the forward strand. The corresponding T to A conversion in the reverse strand is also shown.

FIG. 3. A 71 base pair oligonucleotide (30 micrograms) containing the SCA mutation is introduced into G-CSF mobilized normal CD34+ cells (200,000 in 500 microliters) isolated from peripheral blood (AllCells, Berkeley CA) by electroporation in Iscove's Modified Dulbecco's Medium (IMDM) containing 1% human serum albumin, monothioglycerol (12.5 micrograms per ml), penicillin (25 U/ml), streptomycin (25 micrograms/ml) with a square wave pulse of 38 ms at 300 V. The rate of gene conversion for pooled cells three days after electroporation was 3.3%. Red blood cell (RBC) differentiation was achieved by stimulation with erythropoietin (3U/ml) for two weeks. RBC were fixed and stained with fluorescent labeled anti-sickle and anti-normal human hemoglobin monoclonal antibodies using the Cytofix/Cytoperm Kit from BD Biosciences, CA. FACS analysis shows that 36% of the control (no oligonucleotide, no electroporation) and 41% of the of the experimental cells were reactive against anti-normal (Hb A-FITC) antibody whereas 0.02% of the control and 0.2% of the experimental cells were reactive with the anti-sickle (Hb S) antibody. In this experiment, normal cells are converted to sickle and the converted cells produce sickle hemoglobin.

FIG. 4. Normal CD34+ cells are cultured for 0, 3 or 4 days (prestimulation) before treatment with an FITC-labeled oligonucleotide designed to convert normal cells to sickle. Various voltages are examined and the pulse time was 38 ms in a square wave electroporation device. The rate of gene conversion in CFC colonies cultured for two weeks is 1.3% and 1.0% respectively for cells pre-cultured for 3 or 4 days respectively.

FIG. 5. Oligonucleotide uptake and viability is examined after electroporation in IMDM medium containing no additives. A FAM-labeled oligonucleotide designed to convert normal cells to sickle is introduced by electroporation at 260V for 38 ms using 30 micrograms of oligonucleotide and 200,000 cells in 100 microliters. Viability was approximately 40% and oligonucleotide uptake measured three hours after electroporation is about 90%. Control cells with no oligonucleotide and no electroporation have a viability of about 94%.

FIG. 6. Three days after electroporation in IMDM having no additives, cell pools are lysed and DNA is extracted for PCR amplification, cloning and detection of the conversion of the normal to sickle mutation. Gene conversion is detected by a fluorogenic 5′ nuclease assay using TaqMan allele specific amplification in the presence of two distinct fluorescent-labeled (VIC and FAM) probes complementary to the normal and sickle sequences. The converted sickle mutation is present in 5.7% of the coloned beta-globin sequences tested. The sickle sequence is not detected in amplified DNA from control cells.

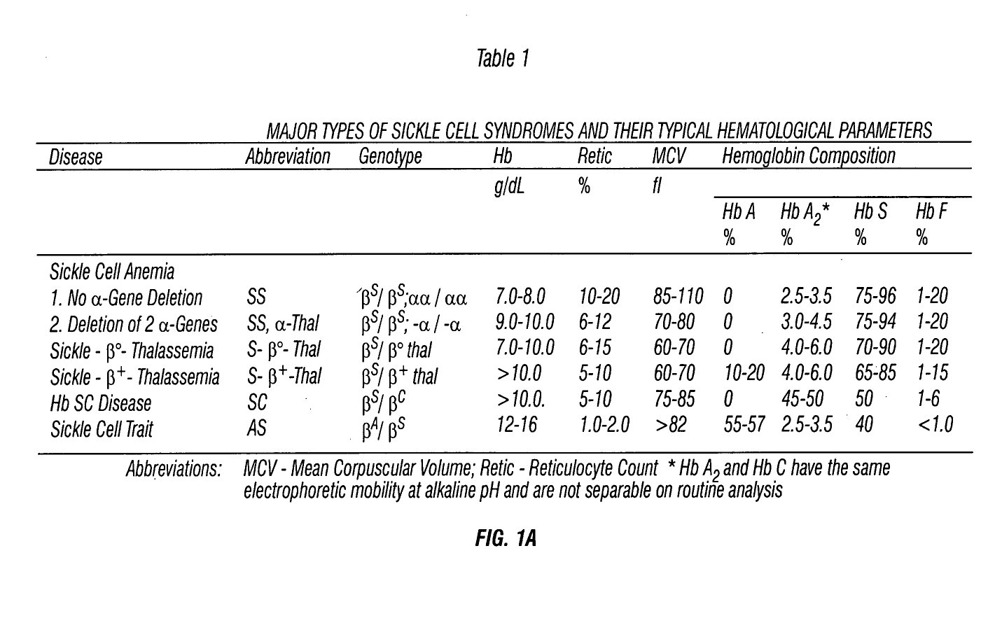

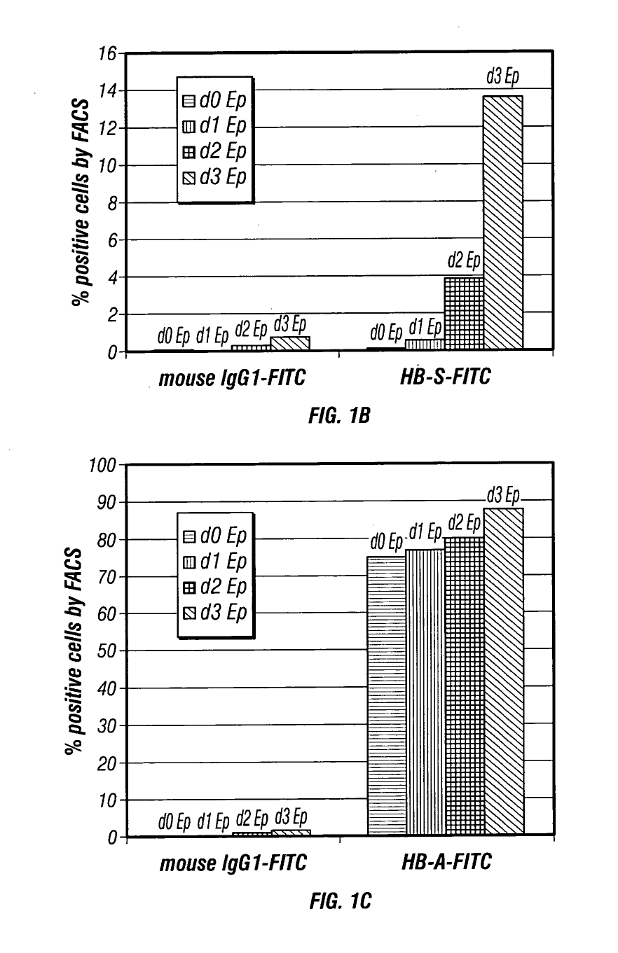

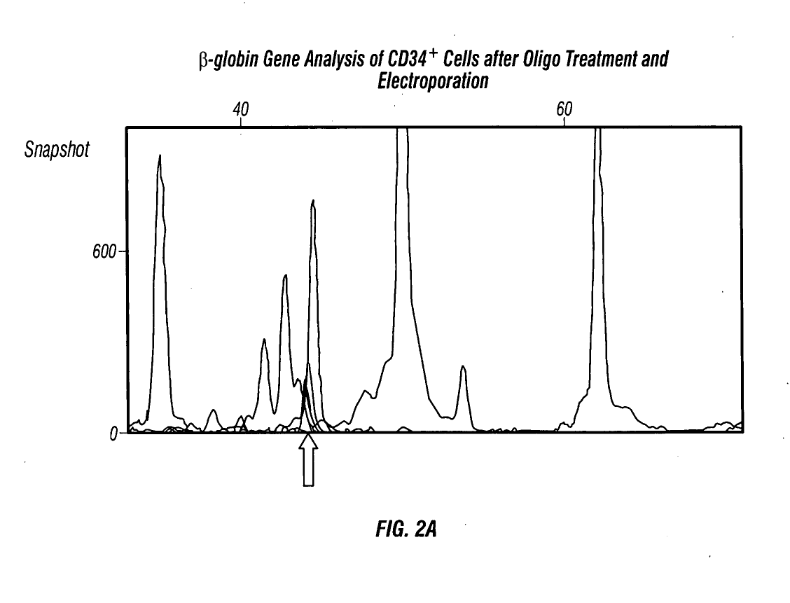

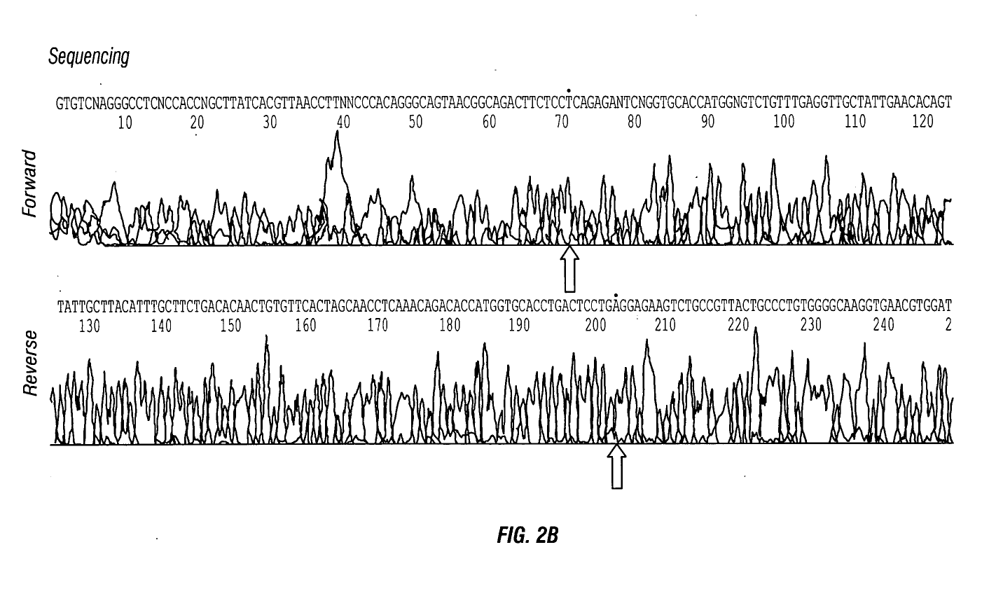

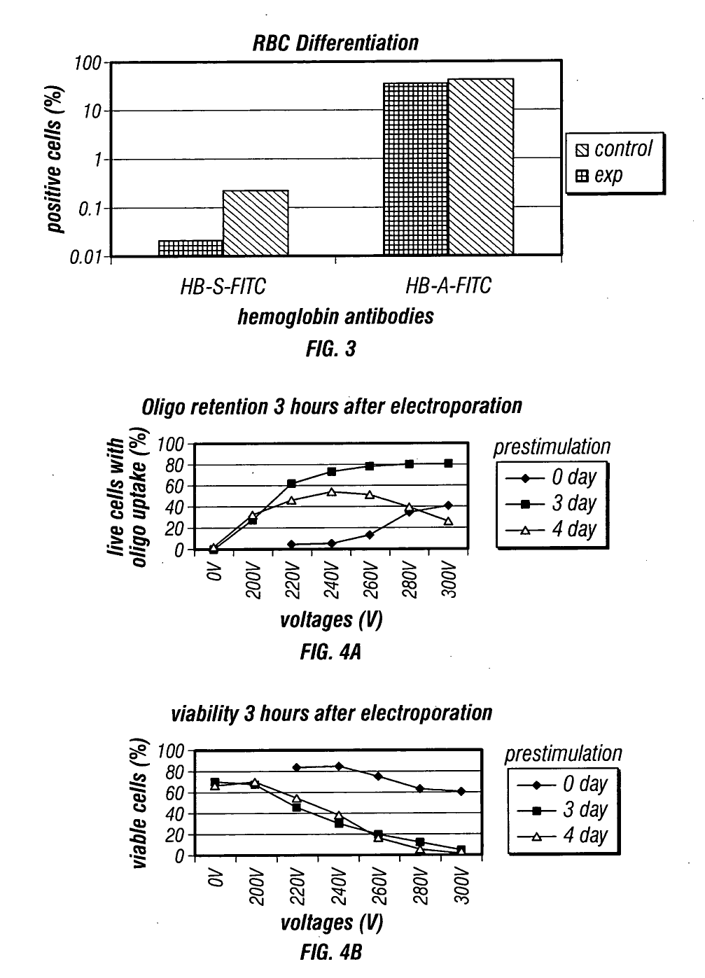

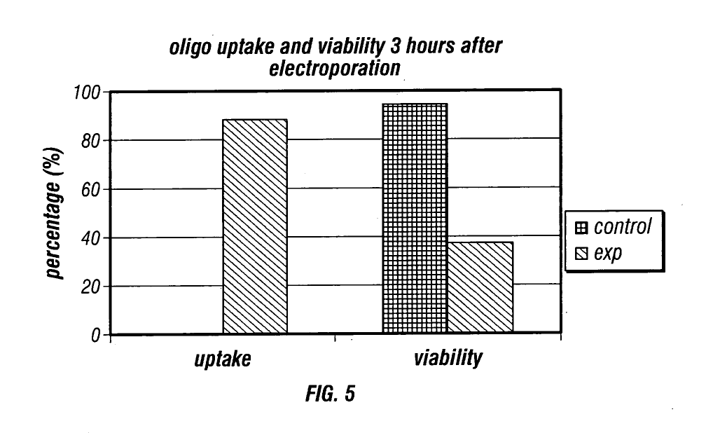

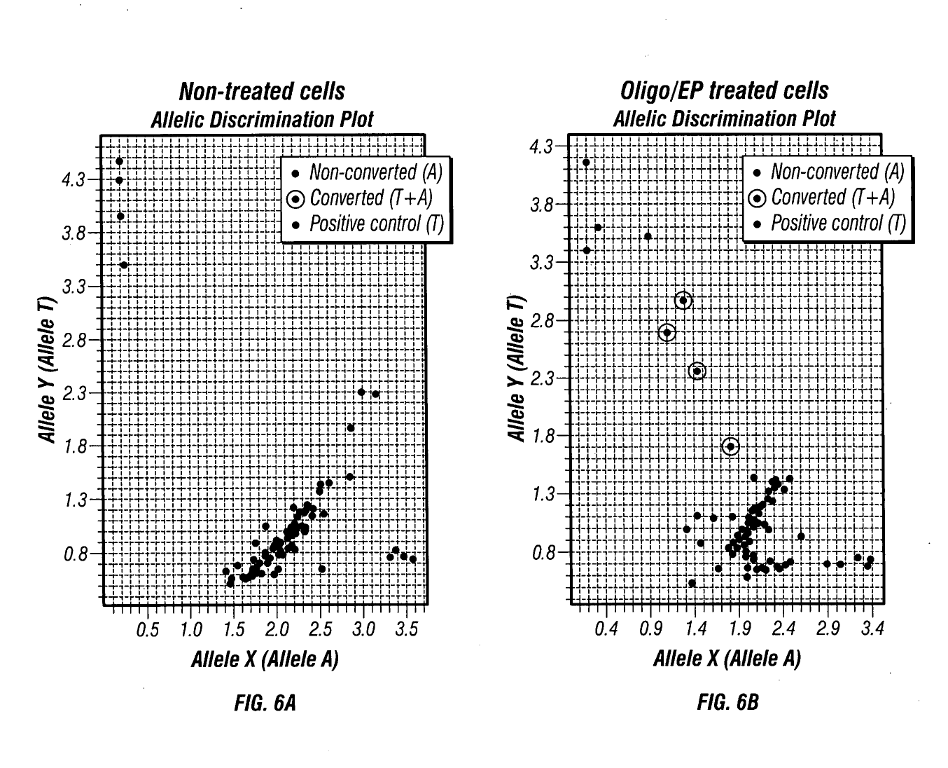

All publications and patent applications or patents cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of producing at least two populations of hemoglobin in a mammal, comprising the steps of:
providing a mammal having a disease-causing globin gene, including a human, with stem/progenitor cells,
wherein an oligonucleotide or polynucleotide introduced into said cells results in a nucleotide alteration of a target globin gene in said cells and non-disease causing hemoglobin is produced in red blood cells differentiated from said stem/progenitor cells.

2. A method of producing at least two populations of hemoglobin in a cell culture, comprising the steps of:
obtaining selectively enriched cells comprising hematopoietic stem/progenitor cells wherein an oligonucleotide or polynucleotide introduced into said cells results in a nucleotide alteration of a target globin gene in said cells; and
demonstrating the production of two populations of hemoglobin in red blood cells following differentiation of the stem/progenitor cells.


SEQUENCE LISTING (2 sequences)

SEQ ID NO: 1 - DNA, 123 bp, Artificial Sequence
  Synthetic polynucleotide sequence
gtgtcnagggcctcnccaccngcttatcacgttaaccttnncccacagggcagtaacggcagacttctcctcagagantcnggtgcaccatggngtctgtttgaggttgctattgaacacagt

SEQ ID NO: 2 - DNA, 124 bp, Artificial Sequence
  Synthetic polynucleotide sequence
tattgcttacatttgcttctgacacaactgtgttcactagcaacctcaaacagacaccatggtgcacctgactcctgaggagaagtctgccgttactgccctgtggggcaaggtgaacgtggat